SEQUENCE LISTING (6 sequences)

SEQ ID NO: 1 - DNA, 49 bp, Artificial Sequence
  synthetic
atagtttagcggccgcgatatctcactaacactcattcctgttgaagct

SEQ ID NO: 2 - DNA, 57 bp, Artificial Sequence
  synthetic
atagtttagcggccgctcactagctagctttaccaggagagtgggagagactcttct

SEQ ID NO: 3 - DNA, 68 bp, Artificial Sequence
  synthetic
ctagcgacatggccaagaaggagacagtctggaggctcgaggagttcggtaggttcacaaacaggaac

SEQ ID NO: 4 - DNA, 71 bp, Artificial Sequence
  synthetic
acagacggtagcacagactatggtattctccagattaacagcaggtattatgacggtaggacatgataggc

SEQ ID NO: 5 - DNA, 70 bp, Artificial Sequence
  synthetic
gctgtaccggttcttcctctgtcagacctccgagctcctcaagccatccaagtgtttgtccttgtgtctg

SEQ ID NO: 6 - DNA, 69 bp, Artificial Sequence
  synthetic
ccatcgtgtctgataccataagaggtctaattgtcgtccataatactgccatcctgtactatccgccgg

(19) United States
(12) Patent Application Publication
(10) Pub. No.: US 2002/0187131 A1
(43) Pub. Date: Dec. 12, 2002

(54) Enhanced antigen delivery and modulation of the immune response therefrom

(76) Inventors: Daniel Hawiger, New York, NY (US); Michel C. Nussenzweig, New York, NY (US); Ralph M. Steinman, Westport, CT (US)

Correspondence Address: KLAUBER & JACKSON, 411 HACKENSACK AVENUE, HACKENSACK, NJ, 07601

(21) Appl. No.: 09/925,284

(22) Filed: Aug. 9, 2001

Related U.S. Application Data
(63) Continuation-in-part of application No. 09/586,704, filed on Jun. 5, 2000.
Application No. 09/586,704 is a continuation of application No. 08/381,528, filed on Jan. 31, 1995, now abandoned.

(51) Int. Cl. A61K 39/395

(52) U.S. Cl. 424/93.7; 424/144.1; 424/178.1

(57) ABSTRACT

Methods are provided for the enhanced delivery of an antigen to antigen-presenting cells such as dendritic cells by conjugating an antigen to a molecule targeted to an endocytic receptor on the dendritic cell, such as DEC-205. The molecule targeted to an endocytic receptor may be a natural ligand to the receptor or an antibody thereto. Conjugates may be covalent complexes or single-chain polypeptides. Together with modulation of the maturation of the dendritic cell, an enhanced immune response or tolerizing immune response may be generated to the antigen. In addition, conjugate molecules including single-chain polypeptides are provided which contain at least an endocytic receptor-binding molecule and a preselected antigen.

### CROSS-REFERENCE TO RELATED APPLICATIONS

This application is a continuation-in-part of Ser. No. 09/586,704, filed Jun. 5, 2000, pending, which is a continuation of Ser. No. 08/381,528, filed Jan. 31, 1995, now abandoned. Both prior applications are incorporated herein by reference in their entireties.

### GOVERNMENT SUPPORT

The research leading to the present invention was supported in part by the Public Health Service grant AI13013. The government may have certain rights in the present invention.

### BACKGROUND OF THE INVENTION

Dendritic cells (DCs) are uniquely potent inducers of primary immune responses in vitro and in vivo (J. Banchereau, R. M. Steinman, Nature 392, 245-52 (1998); C. Thery, S. Amigorena, Curr. Opin. Immunol. 13, 45-51. (2001)). In tissue culture experiments, DCs are typically two orders of magnitude more effective as antigen presenting cells (APCs) than B cells or macrophages (K. Inaba, R. M. Steinman, W. C. Van Voorhis, S. Muramatsu, Proc Natl Acad Sci USA 80, 6041-5 (1983); R. M. Steinman, B. Gutchinov, M. D. Witmer, M. C. Nussenzweig, J Exp Med 157, 613-27 (1983)). In addition, purified, antigen-bearing DCs injected into mice or humans migrate to lymphoid tissues and efficiently induce specific immune responses (M. V. Dhodapkar, et al., J Clin Invest 104, 173-80 (1999); K. Inaba, J. P. Metlay, M. T. Crowley, R. M. Steinman, J Exp Med 172, 631-40 (1990); R. I. Lechler, J. R. Batchelor, J Exp Med 155, 31-41 (1982)). Likewise, DCs migrate from peripheral tissues to lymphoid organs during contact allergy (S. E. Macatonia, S. C. Knight, A. J. Edwards, S. Griffiths, P. Fryer, J Exp Med 166, 1654-67 (1987); A. M. Moodycliffe, et al., J Exp Med 191, 2011-20 (2000)) and transplantation (C. P. Larsen, P. J. Morris, J. M. Austyn, J Exp Med 171, 307-14 (1990)), two of the most powerful, known stimuli of T cell immunity in vivo. Based on these and similar experiments, it has been proposed that the principal function of DCs is to initiate T cell mediated immunity (J. Banchereau, R. M. Steinman, Nature 392, 245-52 (1998)). However, nearly all of these prior art experiments involved DC purification or culture in vitro, or some perturbations in vivo that induce major alterations in DC maturation and function. Thus, the physiologic function of DCs in the steady state has not been determined (K. Inaba, J. P. Metlay, M. T. Crowley, R. M. Steinman, J Exp Med 172, 631-40 (1990); B. Thurner, et al., J Exp Med 190, 1669-78 (1999)).

There is indirect evidence from a number of different laboratories suggesting that DCs may play a role in maintaining peripheral tolerance (summarized in R. M. Steinman, S. Turley, I. Mellman, K. Inaba, J Exp Med 191, 411-6 (2000)). For example, injection of mice with 33D1, a rat monoclonal antibody to an unknown DC antigen, appeared to induce T cell unresponsiveness to the rat IgG (F. D. Finkelman, A. Lees, R. Birnbaum, W. C. Gause, S. C. Morris, J Immunol 157, 1406-14. (1996)). However, the specificity of antigen delivery was uncertain and the relevant T cell responses could not be analyzed directly. In addition, peripheral tolerance to ovalbumin and hemagglutinin expressed in pancreatic islets was found to be induced by bone marrow derived antigen presenting cells (A. J. Adler, et al., J Exp Med 187, 1555-64. (1998); C. Kurts, H. Kosaka, F. R. Carbone, J. F. Miller, W. R. Heath, J Exp Med 186, 239-45. (1997); D. J. Morgan, H. T. Kreuwel, L. A. Sherman, J Immunol 163, 723-7. (1999)), but the identity of these antigen presenting cells has not been determined (W. R. Heath, F. R. Carbone, Annu Rev Immunol 19, 47-64 (2001)).

Co-pending application Ser. No. 09/586,704 describes the endocytic cell membrane receptor DEC-205, which is present on mammalian dendritic cells as well as on certain other cell types, and describes its role in antigen processing, and exploiting the existence of DEC-205 primarily on dendritic cells for targeting antigens for uptake and presentation by dendritic cells. The application describes ligands of DEC-205, such as antibodies, carbohydrates as well as other DEC-205-binding agents for targeting antigens to DEC-205 and thus specifically to dendritic cells.

It is toward the enhancement of antigen delivery to antigen-presenting cells and the manipulation of the immune response resulting therefrom that the present invention is directed.

The citation of any reference herein should not be deemed as an admission that such reference is available as prior art to the instant invention.

### SUMMARY OF THE INVENTION

In its broadest aspect, the present invention is directed to enhancing the delivery of a preselected antigen to an antigen-presenting cell by targeting the preselected antigen to an endocytic receptor on the antigen-presenting cell. A non-limiting but preferred antigen-presenting cell is a dendritic cell (DC). Non-limiting examples of dendritic cell endocytic receptors include DEC-205, the asialoglycoprotein receptor, the Fcγ receptor, the macrophage mannose receptor, and Langerin. A preferred receptor is DEC-205. Enhanced processing and presentation of antigen to T cells is achieved by the foregoing method. The foregoing enhanced presentation by the method of the invention, in combination with other factors or conditions, may lead to a more robust immune response to the preselected antigen, or tolerance to the preselected antigen.

The foregoing enhanced antigen presentation in combination with manipulating the antigen-presenting cell may be carried out in order to modulate the immune response to the preselected antigen delivered via the endocytic receptor. To enhance the development of a cellular immune response to the preselected antigen, delivery of the antigen via the endocytic receptor to a dendritic cell (DC) in combination with DC maturation is carried out. DC maturation may be induced by any means, such as by way of non-limiting examples, CD40 ligation, CpG, ligation of the IL-1, TNF or TOLL receptor, or activation of an intracellular pathway such as TRAF-6 or NF-κB. In a preferred but non-limiting embodiment, DC maturation is achieved by CD40 ligation.

To induce tolerance to the preselected antigen, antigen delivery to a dendritic cell is carried out in the absence of DC maturation, such as the absence of CD40 ligation, or in the absence of any other DC maturation signal such as but not limited to those described above.

The foregoing methods are carried out in an animal in which either an enhanced immune response is desired or a tolerizing immune response is desired, or it may be carried out ex vivo and antigen-presenting cells introduced into the animal. The antigen delivery may be carried out ex vivo, using antigen-presenting cells isolated from the animal, after which the cells may be optionally isolated and returned to the animal. Subsequently, in-vivo manipulation of DC maturation, such as by CD40 ligation, is carried out to direct the immune response to the desired outcome. Alternatively, both the antigen exposure and DC maturation or inhibition of DC maturation may be carried out ex vivo before optional isolation of antigen-presenting cells and introduction into the animal. In yet another embodiment, both antigen delivery and manipulation of DC maturation may be carried out in vivo.

Various routes of delivery are embraced herein, including but not limited to parenteral or transmucosal delivery, e.g., orally, nasally, or rectally, or transdermally. Parenteral includes but is not limited to, intra-arterial, intramuscular, intradermal, subcutaneous, intraperitoneal, intraventricular, and intracranial administration. Pulmonary, intraintestinal, and delivery across the blood brain barrier are also embraced herein.

Administration as a vaccine for enhancement of an immune response is a preferred embodiment.

Delivering the preselected antigen to the endocytic receptor is carried out by exposing the antigen cell to a conjugate or complex between a molecule that binds the endocytic receptor, and the antigen. In the instance where the endocytic receptor is DEC-205, the method is carried out by exposing the antigen-presenting cell to a conjugate that includes both a DEC-205-binding molecule and a preselected antigen. As will be seen below, the antigen may be any compound, molecule, or substance desirably enhancedly delivered to an antigen-presenting cell, such as a protein, peptide, carbohydrate, polysaccharide, lipid, nucleic acid, cell, by way of non-limiting examples. Various means of conjugating or complexing the antigen to the endocytic receptor-binding molecule is embraced herein, including but not limited to covalent cross-linking, and in the instance where both molecules are proteins or peptides, expression together in a single-chain polypeptide.

In the instance where the endocytic receptor is DEC-205, the DEC-205-binding molecule may be any ligand for DEC-205, including antibodies or natural ligands. In a preferred embodiment, the DEC-205-binding agent is an antibody, and most preferably a monoclonal antibody, such as but not limited to NLDC-145. However, natural ligands to DEC-205 may be utilized, examples of which are described herein, wherein conjugation or covalently coupling the preselected antigen thereto is also embraced by the present invention.

The antigen may be any compound, substance or agent for which a modulated immune response is desired or for which enhanced delivery into antigen-presenting cells is desired. Such antigens may include proteins, cells, nucleic acids including DNA, RNA, and antisense oligonucleotides, carbohydrates, polysaccharides, lipids, glycolipids, among others. Non-limiting examples include immunogenic portions of HIV-1, HPV, EBV, HSV, Mycobacterium tuberculosis, and malaria, for use in a vaccine to enhance the development of an immune response thereto. In the instance where tolerization to an antigen is desired in order to prevent or piophylax toward a potential immune response, such antigens include transplant antigens, allergens and autoimmune antigens, by way of non-limiting example.

To enhance the development of an immune response to the antigen delivered via the DEC-205 receptor, DC maturation or exposure of the DC to a maturation signal may be achieved in any of a number of ways. In the example in which CD40 ligation is used, it may be achieved by exposing the antigen-presenting cell ex vivo or in vivo to an agonistic anti-CD40 antibody, although other methods and agents for achieving CD40 ligation are embraced herein. Exposure of DCs to other maturation signals in the form of agonistic antibodies to other receptors is embraced herein. Activation of intracellular DC maturation signals may be achieved by, for example, by ligands that signal Toll like receptors, e.g., CpG oligodeoxynucleotides, RNA, bacterial lipoglycans and polysaccharides, TNF receptors such as the TNFα receptor, IL-1 receptors, and compounds that activate TRAF 6 or NF-κB signaling pathways. Both natural ligands for DEC-205 as well as antibodies may be used.

In another embodiment, a method is provided for enhancing the development of tolerance to a preselected antigen by delivering the preselected antigen to a DEC-205 receptor on an antigen-presenting cell having a DEC-205 receptor in the absence of DC maturation. Methods and conjugates for delivering the antigen are as described above. Non-limiting examples of antigens for which tolerance of the immune system is desirable include transplant antigens, allergens, and antigens toward which autoimmunity has or may develop. In one embodiment, the use of ligands that are recognized by the C-type lectin and other domains of the DEC-205 receptor, including such modifications of vaccines that are recognized by DEC-205 receptor, such as modified tumor cells and tumor antigens, microbial vectors and associated antigens, and autoantigens.

The present invention is also directed to conjugates between an antigen-presenting cell endocytic receptor-binding molecule and a preselected antigen for the aforementioned purposes, and pharmaceutical compositions comprising such conjugates. Non-limiting examples of the antigen-presenting cell is a dendritic cell, and of endocytic receptors, DEC-205, the asialoglycoprotein receptor, the Fcγ receptor, the macrophage mannose receptor, and Langerin. As noted above, the conjugates may be a covalently cross-linked or a conjugate between the receptor-binding molecule and a preselected antigen. The antigen may be any material, substance or compound for which enhanced delivery to an antigen-presenting cell, such as dendritic cell is desired, including but not limited to proteins, cells, nucleic acids such as DNA and RNA, carbohydrates, etc. In the embodiment wherein the preselected antigen is a peptide antigen or a protein antigen, and the endocytic receptor-binding molecule is a protein, such as an antibody or protein ligand, the antigen and the binding protein may reside on the same polypeptide chain. In a preferred embodiment, the endocytic receptor is DEC-205, and the DEC-205-binding protein is an antibody. In another embodiment, the antigen is recognized directly by the DEC-205 multilectin receptor.

The invention is also directed to polynucleotides encoding the aforementioned single-chain chimeric polypeptides.

As noted above, the enhanced delivery of molecules to an antigen-presenting cell such as a dendritic cell is achieved by coupling the molecule to, for example, a DEC-205-targeting agent. In addition to enhanced antigen delivery, targeting of nucleic acids to antigen-presenting cells via an endocytic receptor such as DEC-205 is a means for introducing foreign DNA into an antigen-presenting cell for transfection or other gene therapy purposes. It need not be associated with DC maturation or absence of DC maturation thereof to achieve this embodiment of the invention.

Other antigen-presenting cell endocytosis receptors other than DEC-205 are likewise targets for enhanced antigen-presenting cell delivery, such as but not limited to the asialoglycoprotein receptor, the Fcγ receptor, the macrophage mannose receptor, and Langerin. All of the aforementioned uses of DEC-205, and compositions comprising a DEC-205-targeted molecule and an antigen respectively pertain to other endocytosis receptors.

It is thus an object of the invention to provide a method for enhancing the development of a cellular immune response to a preselected antigen comprising delivering the preselected antigen to an endocytic receptor on a dendritic cell and inducing promoting maturation of the dendritic cell. In one embodiment, the endocytic receptor is DEC-205. The delivering of the preselected antigen to DEC-205 may include at least exposing the dendritic cell to a DEC-205-binding agent comprising the preselected antigen. The DEC-205-binding agent including at least the preselected antigen may be a conjugate between said DEC-205-binding agent and said preselected antigen. In a preferred embodiment, the preselected antigen may be a peptide antigen or a protein antigen, and the peptide or protein antigen may be conjugated to the DEC-205-binding agent by means of a cross-linking agent.

In the instance where the DEC-205-binding agent is a protein, it is a further object of the invention to provide a DEC-205-binding agent and a peptide antigen or protein antigen on a single polypeptide chain. In a preferred embodiment, the DEC-205-binding agent may be an antibody.

It is a further object of the invention to enhance the development of an immune response to the antigen by inducing maturation of the dendritic cell with CD40 ligation. CD40 ligation may be achieved by exposing the dendritic cell to an agonistic anti-CD40 antibody. The delivering of the preselected antigen to DEC-205 and promoting dendritic cell maturation in the dendritic cell may be independently carried out ex vivo or in vivo.

It is yet a further object of the invention to provide a method for enhancing the development of tolerance to a preselected antigen by at least delivering the preselected antigen to an endocytic receptor on a dendritic cell in the absence of dendritic cell maturation. The endocytic receptor may be DEC-205. The delivering of the preselected antigen to the DEC-205 may be carried out by at least exposing the dendritic cell to a DEC-205-binding agent that contains the preselected antigen. The DEC-205-binding agent that contains the preselected antigen may be a conjugate between the DEC-205-binding agent and the preselected antigen. In the case in which the preselected antigen is a peptide antigen or a protein antigen, the conjugate of the DEC-205-binding agent may be by means of a cross-linking agent. Where the DEC-205-binding agent is a protein, the DEC-205-binding agent and the peptide antigen or protein antigen may be present on a single polypeptide chain. In a preferred embodiment, the DEC-205-binding agent may be an antibody. In the foregoing method, agents that block intracellular signalling at the levels of TRAF 6 and NF-κB, which are used by CD40 and Toll-like receptors and IL-1r to trigger dendritic cell maturation.

It is still yet a further object of the invention to provide a conjugate for enhanced delivery of a preselected antigen to a dendritic cell, the conjugate being at least a covalent complex between a binding molecule to an endocytic receptor and the antigen. The endocytic receptor may be DEC-205. The binding molecule to DEC-205 may be an antibody to DEC-205. In one embodiment, the antigen may be covalently bound to the antibody to DEC-205 via a cross-linking agent. The antigen may be a peptide or a protein. In one embodiment, the peptide or protein and a light chain or a heavy chain of the antibody to DEC-205 may reside on the same polypeptide chain, forming a chimeric polypeptide.

It is another object of the invention to provide polynucleotides that encode the chimeric polypeptides mentioned above.

It is yet still an even further object of the invention to provide a method for enhancing the delivery of a preselected antigen to a dendritic cell by at least exposing the dendritic cell to the conjugate or chimeric polypeptide described above. Non-limiting examples of the foregoing antigens include a protein, cell, nucleic acid, carbohydrate, polysaccharide, lipid, or glycolipid. The nucleic acid may be DNA, RNA or an antisense oligonucleotide.

These and other aspects of the present invention will be better appreciated by reference to the following drawings and Detailed Description.

### BRIEF DESCRIPTION OF THE DRAWINGS

FIGS. 1 A-E show that the monoclonal antibody NLDC-145 targets DCs in vivo.

FIGS. 2 A-B show that DCs process and present antigen delivered by hybrid antibodies comprising amino acids 46-61 of hen white lysozyme added to the carboxy terminus of cloned NLDC145 monoclonal antibody to DEC-205 (αDEC/HEL).

FIGS. 3 A-E demonstrate in-vivo activation of CD4⁺ T cells by αDEC/HEL.

FIGS. 4 A-C shows that CD4⁺ T cells divide in response to antigen presented by DCs in vivo, produce Il-2 but not IFN γ, and are then rapidly deleted.

FIGS. 5 A-C show that CD40 ligation prolongs T cell activation in response to antigens delivered to DCs and induces up-regulation of co-stimulatory molecules on DCs.

### DETAILED DESCRIPTION OF THE INVENTION

The inventors herein have found that enhanced antigen delivery to antigen-presenting cells may be achieved by targeting the antigen to a DC-restricted endocytic receptor, such as DEC-205, the asialoglycoprotein receptor, the Fcγ receptor, the macrophage mannose receptor, and Langerin. Furthermore, manipulating the environment of the thus-targeted antigen-presenting cell with regard to dendritic cell maturation can dictate the outcome of the endocytic receptor-targeted enhanced antigen presentation: towards eliciting a potent cellular immune response, or, alternatively, tolerance of the immune system to the endocytic receptor-targeted antigen. A preferred antigen-presenting cell is a dendritic cell (DC), and a preferred endocytic receptor is DEC-205. As will be seen below, the antigen may be targeted to the DEC-205 receptor on dendritic cells by any of a number of means, such as by conjugating or complexing the antigen to a DEC-205 ligand such as an antibody to DEC-205, or utilizing a fusion protein which is a hybrid of an anti-DEC-205 antibody and the antigen, if the antigen is a protein or peptide. Both exposure to the targeted antigen and manipulation of DC maturation in the environment of the antigen-presenting cell may be independently performed ex vivo or in vivo. Manipulation of DC maturation includes exposing or not exposing the antigen-presenting cells to a CD maturation stimulus such as a CD40 ligation promoting agent(s), or exposing the antigen-presenting cells to an agent which abrogates a DC maturation stimulus such as CD40 ligation, the latter in order to achieve an environment in which DC maturation does not occur.

Dendritic cells (DCs) have the capacity to initiate immune responses, but it has been postulated that they may also be involved in inducing peripheral tolerance. As will be seen in the examples below, to examine the function of DCs in the steady state, the present inventors devised an antigen delivery system targeting these specialized antigen presenting cells in vivo using a monoclonal antibody to the DC-restricted endocytic receptor, DEC-205. The results show that this route of antigen delivery to DCs is several orders of magnitude more efficient than free peptide in Complete Freund's Adjuvant (CFA) in inducing T cell activation and cell division. However, T cells activated by antigen delivered to DCs in this fashion without more are not polarized to produce Th1 cytokine IFN-γ and the activation response is not sustained. Within 7 days the number of antigen-specific T cells is severely reduced, and the residual T cells become unresponsive to systemic challenge with antigen in CFA. Thus, without dendritic cell stimulation at the time of antigen presentation, tolerance to the delivered antigen rather than induction of a cellular response is achieved. In contrast, co-injection of the DC-targeted antigen with anti-CD40 agonistic antibody changes the outcome from tolerance to prolonged T cell activation and immunity.

While co-pending application Ser. No. 09/586,704 exploited the restriction of the DEC-205 receptor molecule to dendritic cells as a means for targeted DC delivery, it was not appreciated until the studies described herein of the several orders of magnitude increased efficiency of antigen delivery by the DEC-205 route as compared to other routes of antigen delivery to dendritic cells, nor was it known that the induction of tolerance could be achieved by targeted delivery of an antigen through an endocytic receptor such as DEC-205 in concert with the absence of CD40 ligation. While the examples below are focused on DEC-205 as the DC receptor for targeting and enhanced uptake thereof, other DC endocytic receptors such as the asialoglycoprotein receptor, the Fcγ receptor, the macrophage mannose receptor, and Langerin, are embraced herein, and all utilities of DEC-205 are applicable to this as well as other endocytic receptors. Moreover, while enhanced antigen presentation by antigen-presenting cells to T cells is a desirable goal achieved herein, enhanced targeting to DC of any substance or molecule is embraced herein, such as enhanced genetic manipulation of DC by targeting a polynucleotide thereto for genetic modification including transfection or antisense therapy. These other aspects of the invention are fully embraced herein.

Exposing antigen-presenting cells to the DEC-205-targeted antigen and any of the foregoing DC maturation stimuli or maturation-inhibiting factors may be achieved in a variety of ways, for example, by exposing isolated antigen-presenting cells ex vivo to the targeted antigen before returning them to the animal, and then no administration to the animal of any factors, or administration of a DC maturation factor, such as, in the case of CD40 ligation, of an anti-CD40 agonistic antibody, or administration to the animal of a factor that will inhibit CD40 ligation in vivo. Alternatively, both antigen exposure and manipulation of CD40 ligation may be performed ex vivo before the antigen-presenting cells are optionally isolated and then readministered to the animal. These and other variations in the protocols are fully embraced by the invention herein, which in this embodiment essentially combines DEC-205-targeted antigen delivery with manipulation of CD40 ligation to modulate the immune response to the antigen. As noted above, the combination of any other endocytic receptor-binding molecule and any other DC maturation stimulus or factor to achieve an enhanced immune response is fully embraced by the teachings herein.

Various routes of delivery are contemplated for an in-vivo administered therapy as described herein. One of the purposes of DC delivery plus DC maturation is to enhance an immune response to a particular antigen, and the methods of the invention achieve such a goal by a vaccination protocol using an immunogen conjugated to a DC-targeted molecule, and co-administration of a DC maturation stimulus, is described herein. Such conjugates, as well as DC maturation stimuli (or inhibitors thereof for the induction of tolerance), may be delivered to the body by any appropriate route for the particular antigen involved. Such routes may include administration parenterally, transmucosally, e.g., orally, nasally, or rectally, or transdermally. Parenteral administration includes intravenous injection, intra-arterial, intramuscular, intradermal, subcutaneous, intraperitoneal, intraventricular, and intracranial administration. Pulmonary delivery is also embraced, as are means for achieving delivery across the blood brain barrier. Intra-intestinal immunization may be achieved by delivery to the immune cells of the intestinal tract. Various formulations of the conjugate, including sustained release formulations, in order to achieve the optimal immunization protocol for the intended goal of the immunogen, are fully embraced herein. Targeting the conjugate on DEC-205 on brain endothelium is another means for achieving the delivery of the antigen across the blood brain barrier.

DEC-205 is described in co-pending application Ser. No. 09/586,704, and incorporated herein by reference in its entirety. Any means for targeting an antigen or antigenic fragment thereof to the DEC-205 receptor on dendritic or other antigen-presenting cells is embraced by the present invention. For example, an antibody to DEC-205 may be used, and the antigen or antigenic fragment thereof conjugated to the antibody using a cross-linking agent. In another embodiment, the antigen or fragment thereof may be part of a chimeric or fusion polypeptide comprising the antibody to DEC-205, wherein a polynucleotide encoding both the antibody to DEC-205 and the fragment reside on the same polynucleotide construct, and are expressed in the form of the chimeric, single-chain antibody-antigen. The antigen may be located at any site in the antibody where it does not interfere with the targeting of the chimeric antibody-antigen to the DEC-205; by way of non-limiting example, appending the antigen to the C-terminus of the antibody heavy chain achieves this purpose. In another embodiment, a DEC-205 targeted composition of the invention may comprise a protein or peptide DEC-205 ligand other than an antibody, and a protein or peptide antigen, residing on the same polypeptide chain. Polynucleotides encoding the aforementioned chimeric polypeptide are also embraced herein.

One non-limiting example of a monoclonal antibody to DEC-205 that may be used in the present invention is NLDC-145, as described in G. Kraal, M. Breel, M. Janse, G. Bruin, J Exp Med 163, 981-97 (1986). However, the invention is not so limited and any antibody may be used, directed to the DEC-205 of the species of animal in which immune therapy by the methods herein is to be achieved. Preferably, the DEC-205-binding molecule binds to human DEC-205.

In another embodiment, a bispecific antibody may be provided, one antigen-binding site directed to DEC-205, and the other antigen-binding site directed to the antigen selected for manipulation of the immune response. This embodiment is particularly useful if an endogenous antigen, such as a cancer antigen, is desirably chosen for enhancing an immune response thereto: administration of the bispecific antibody to the patient exhibiting circulating levels of the cancer antigen will target it to the dendritic cells, which, in combination with the manipulation of CD40 ligation as described herein, will result in an enhanced anti-cancer antigen immune response.

In a further embodiment, if any antibody method is used for the targeting of the antigen to DEC-205, binding of the antibody to the Fc receptor is desirably minimized. To minimize such binding, a recombinant antibody used herein may be modified such as to alter the Fc region of the antibody molecule to reduce its recognition by the Fc receptor. Such modifications have been described (R. A. Clynes, T. L. Towers, L. G. Presta, J. V. Ravetch, Nat Med 6, 443-6 (2000)), and this and other modifications of the conjugate of chimeric DEC-205-binding molecule and the antigen to increase its specificity for binding to the DEC-205 receptor are full embraced herein.

Natural ligands for DEC-205 or the other endocytic receptors described herein may also be used as an alternative to an antibody to the receptor to enhance the delivery of an associated antigen. Other ligands may be identified as described in co-pending applications Ser. Nos. 09/586,704, 08/381,528, as well as in PCT/US96/01383 (WO9623882).

Exploitation of the antigen-presenting cell endocytic receptor for enhanced antigen delivery, with or without subsequent manipulation of DC maturation for modulation of an immune response, may be utilized for antigen delivery and modulation of an immune response in any mammalian species, preferably human but not so limiting, and may be used in non-human primates, livestock and companion animals, zoo animals, as well as animals in the wild. Vaccination by the methods and using the agents herein of domestic or livestock animals against pathogens such as foot and mouth disease, rabies, distemper, among a large number of important pathogens and parasites, is fully embraced herein. Vaccination of humans against viral, bacterial, protistan and multicellular parasitic diseases is also fully embraced herein, including but not limited to HIV-1, human papillomavirus, Epstein-Barr virus, herpes simplex virus, measles virus, smallpox virus, chicken pox virus, the various hepatitis viruses, rubella virus, mumps virus, infectious bacterial agents including pneumococci, tuberculosis, Borrelia burgdorferi, the causative agent of Lyme disease, and diphtheria, among others. Protistan antigens include malaria and trypanosomatids. Multicellular parasites include schistosomes, roundworms, and others. The foregoing are merely non-limiting examples of antigens and diseases associated therewith, and the invention herein embraces all such antigens for the purposes described.

The selection of antigen for enhanced DC delivery and modulation of the immune response thereto may be any antigen for which either an enhanced immune response is desirable, or for which tolerance of the immune system to the antigen is desired. In the case of a desired enhanced immune response to a particular antigen, antigens such as infectious disease antigens for which a protective immune response may be elicited are exemplary. In addition to the infectious and parasitic agents mentioned above, another area for desirable enhanced immunogenicity to a non-infectious agent is in the area of dysproliferative diseases, including but not limited to cancer, in which cells expressing cancer antigens are desirably eliminated from the body. Cancers, particularly metastatic cancers, include but are not limited to prostate, breast, ovarian, testicular, melanoma, as well as many other cancer types. The antigen conjugated or coupled to an endocytic receptor-binding molecule may be a cancer cell, or immunogenic materials isolated from a cancer cell, such as membrane proteins.

The antigen may be a portion of an infectious agent such as HZV-1, EBV, HBV, malaria, or HSV, by way of non-limiting examples, for which vaccines that mobilize strong T-cell mediated immunity (via dendritic cells) are needed.

The antigen may be any molecule or substance for enhanced DC delivery, not only for the immunologic modulation purposes herein but additionally, for example, to promote or enhance the delivery of agents to dendritic cells. In one example, genetic manipulation of dendritic cells may be achieved by targeting a polynucleotide to a dendritic cell via an endocytic receptor such as DEC-205. The polynucleotide may be DNA, RNA, or an antisense oligonucleotide, by way of non-limiting examples. Such a procedure increases the amount of a molecule desirably introduced into a dendritic cell by taking advantage of the enhanced uptake when a molecule is associated with or conjugated to a ligand for or other means of targeting the molecule to DEC-205 or another endocytic receptor. Although the cell may be further manipulated after the delivery, such as maturation or lack thereof, the enhanced delivery aspect of the invention is not necessarily associated with any further manipulation of the dendritic cells. For example, the cells may be removed from the body, a conjugate exposed thereto to deliver the molecule, such as an antisense oligonucleotide or a polynucleotide construct for gene therapy, and the dendritic cells reintroduced to the body. This example is merely illustrative of this aspect of the invention and is in no way limiting.

Attachment of the antigen, or other molecule desirably introduced into a dendritic cell, to the DEC-205- or other endocytic receptor-binding agent may be by any suitable means, including but not limited to covalent attachment by means of a bifunctional cross-linking reagent, and activation of one member and then cross-linking to a functional group on the other. Various cross-linking agents and functional group activating agents such as described from Pierce Chemical Co., Rockford, Ill., are useful for these purposes. In the instance wherein both the endocytic receptor-binding molecule and the antigen are proteins or peptides, they may be expressed on a single polypeptide chain, wherein the single polypeptide chain retains the endocytic receptor-binding activity and the protein or peptide antigen retains its desired features. In one non-limiting example, the endocytic receptor-binding molecule is an DEC-205-binding molecule such as a monoclonal antibody to DEC-205, and one chain of the antibody and the antigen are provided in a recombinant polynucleotide construct in which the expressed polypeptide comprises both an antibody chain with a DEC-205 binding site, and the antigen.

In contrast to a desired enhanced immune response to an antigen, in many instances a lack of an immune response is desired to a particular antigen. By way of non-limiting example, an individual who is a candidate for a transplant from a non-identical twin may suffer from rejection of the engrafted cells, tissue or organ, as the engrafted antigens are foreign to the recipient. Prior tolerance of the recipient individual to the intended engraft abrogates or reduces later rejection. Reduction or elimination of chronic anti-rejection therapies is achieved by the practice of the present invention. In another example, many autoimmune diseases are characterized by a cellular immune response to an endogenous or self antigen. Tolerance of the immune system to the endogenous antigen is desirable to control the disease. In a further example, sensitization of an individual to an industrial pollutant or chemical, such as may be encountered on-the-job, presents a hazard of an immune response. Prior tolerance of the individual's immune system to the chemical, in particular in the form of the chemical reacted with the individual's endogenous proteins, may be desirable to prevent the later occupational development of an immune response. Allergens are other antigens for which tolerance of the immune response thereto is desirable. Likewise, autoantigens could be delivered to dendritic cells by a way that elicits specific immunotolerance.

The invention is directed not only to the use of the aforementioned DEC-205-binding molecules such as anti-DEC-205 antibody conjugates or fusion proteins comprising an antigen, but also to compositions comprising such conjugates of chimeric proteins, and pharmaceutical compositions comprising them, for vaccination or other immune modulation of an animal, preferably a human but any mammalian animal. It also embraces polynucleotide sequences encoding chimeric or single-chain polypeptides comprising an antigen-presenting cell endocytic receptor-binding molecule, such as a DEC-205-binding molecule, and an antigen, The DEC-205-binding molecule may be an antibody, a DEC-205-binding protein, a lectin, or any DEC-205-binding fragment of any of the foregoing.

Alternatively, non-antibody means for targeting an antigen to an endocytic receptor such as DEC-205 may be used, such as those described in co-pending application Ser. No. 09/586,704. Such targeting molecules include a carbohydrate ligand, such as a glycan, that binds to DEC-205, in particular to one of its lectin domains. DEC-205 is known to possess about ten C-type lectin domains, and any or a combination of these domains may serve as targets for specific binding of an antigen to DEC-205. Moreover, other dendritic cell endocytic receptors other than DEC-205, such as but not limited to the asialoglycoprotein receptor, the Fcγ receptor, the macrophage mannose receptor, and Langerin, may be used in a likewise fashion as DEC-205 described herein.

In concert with delivery of the antigen to DEC-205 on the antigen-presenting cell, a DC maturation stimulus or inhibition thereof, such as is achieved by manipulation of CD40 ligation of the antigen-presenting cell, is desirable to achieve the desired immune response outcome. As mentioned above, in concert with CD40 ligation, a robust cellular immune response toward the antigen is achieved. In the absence of CD40 ligation, tolerance to the antigen is achieved. The present invention embraces all such manipulations of CD40 ligation in concert with DEC-205 antigen targeting for the purposes herein. Moreover, the combination of any DC maturation signal and any endocytic receptor-targeted antigen delivery is embraced by the present invention.

DC maturation may be achieved by any one of a number of means, or combinations thereof. Such maturation signals may be achieved by, for example, CD40 ligation, CpG oligodeoxyribonucleotides, ligation of the IL-1, TNFα or TOLL-like receptor, bacterial lipoglycans and polysaccharides or activation of an intracellular pathway such as TRAF-6 or NF-κB. These are merely illustrative and one of skill in the art will be aware of other means for inducing DC maturation, all of which are embraced herein in combination with endocytic receptor delivery of a preselected antigen.

In a preferred but non-limiting embodiment, CD40 ligation may be achieved using any of a number of methods. Exposure of the antigen-presenting cell to an agonistic anti-CD40 antibody achieves CD40 ligation. An antibody such as but not limited to FGK 45 described herein may be used. The invention embraces polyclonal antibodies, monoclonal antibodies, chimeric antibodies, antibody fragments such as F(ab) fragments, and any antibody fragments or recombinant antibody fragments or constructs comprising an antigen-binding site. CD40L or a CD40-binding fragment thereof may be used, such as described in C. Caux, et al., J Exp Med 180, 1263-72 (1994); K. Inaba, et al., J Exp Med 191, 927-36 (2000) and F. Sallusto, A. Lanzavecchia, J Exp Med 179, 1109-18 (1994), by way of non-limiting examples. Ligands that signal Toll like receptors, e.g., CpG oligodeoxynucleotides, RNA, bacterial lipoglycans and polysaccharides, TNF receptors such as the TNFα receptor, IL-1 receptors, and compounds that activate TRAF 6 and NF-κB signaling pathways, may be used.

As mentioned above, to achieve an enhanced immune response, a DC maturation stimulus such as CD40 ligation is desired, as may be achieved by exposing the antigen-presenting cells ex vivo or in vivo to an aforementioned DC maturation signal. In contrast, to tolerize the animal to a DEC-205-targeted antigen, the absence of DC maturation is necessary. This may be achieved ex vivo or in vivo. Agents that block DC maturation signals such as CD40 ligation may be used, such as but not limited to an antibody to CD40L, the TNF-family member that is expressed on activated CD4 T cells, platelets and mast cells, or a soluble CD40 or fragment thereof capable of binding CD40L and inhibiting dendritic cell maturation. Blockage of any of the DC maturation signals mentioned throughout herein, which are merely exemplary, may be performed in concert with endocytic receptor-mediated antigen delivery to achieve the desired tolerance to the antigen. Other means of preventing or inhibiting DC maturation are fully embraced herein.

The methods of the invention may be carried out ex vivo or in vivo, and independently with regard to antigen targeting to an endocytic receptor, such as DEC-205 in the following examples, and manipulation of DC maturation, such as by CD40 ligation manipulation, in the following examples. For fully ex vivo methods, dendritic cells may be isolated from whole blood of an individual, and exposed ex vivo both to the DEC-205-targeted antigen and to CD40 ligation, or in the absence of CD40 ligation, before the dendritic cells are optionally isolated and then readministered to the individual. In another embodiment, isolated dendritic cells are exposed to DEC-205-targeted antigen and then optionally isolated before administration to the individual. Subsequent to readministration, CD40 ligation is manipulated, for example, by no additional steps (to induce tolerance), by administration of a CD40 ligation promoting agent(s) such as an agonistic anti-CD40 antibody for enhancing the development of a cellular response, or for tolerance, a CD40 ligation inhibiting agent, as mentioned above. Routes of in-vivo administration are described hereinabove.

In vivo methods are also included, wherein the DEC-205-targetet antigen is administered to the individual, such as in the form of a vaccine, and then CD40 ligation is manipulated in vivo, by any of the foregoing methods. Route of administration of the vaccine are as described above. Administration of a DC maturation signal may also be performed in vivo, systemically or locally, and via any suitable route of administration.

As mentioned above, the present invention embraces DEC-205-targeted antigen compositions, such as but not limited to a chimeric anti-DEC-205 antibody comprising an antigen, or a conjugate of an aforementioned antibody and an antigen. It is further directed to other DC endocytic receptor-targeted antigens, such as an antigen conjugated to an asialoglycoprotein receptor-targeted molecule.

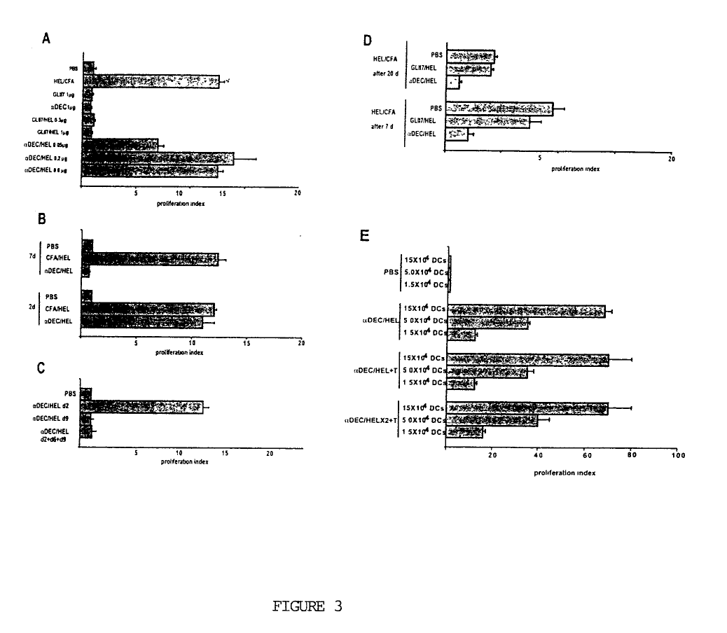

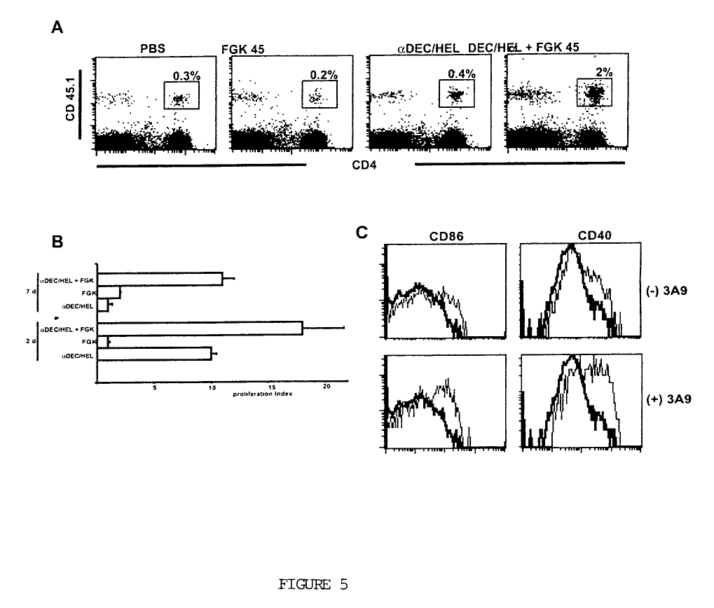

As will be shown in the examples below, manipulation of the environment of the antigen-presenting cell governs whether a tolerance or induced immune response is achieved. When DCs are charged with antigen in the steady state, these MHC II-rich cells do not induce normal Th-subset polarization or prolonged T cell expansion and activation. Instead, the T cells exposed to antigen on DCs in vivo either disappear or become anergic to antigenic re-stimulation. This indicates that in the steady state, the primary function of DCs is to maintain peripheral tolerance (see FIGS. 3C and 3D). Indeed, combined administration of DC-targeted antigen with an agonistic anti-CD40 antibody that up-regulates co-stimulatory molecules like CD86 on the surface of DCs (see FIG. 5C), prevents induction of peripheral tolerance and leads to prolonged T cell activation.

Furthermore, it will be shown that a covalent complex between an antigen, e.g., ovalbumin, and an anti-DEC-205 antibody efficiently targets the MHC I pathway and leads to profound tolerance of CD8 T cells to the antigen.

The following examples are presented in order to more fully illustrate the preferred embodiments of the invention. They should in no way be construed, however, as limiting the broad scope of the invention.

### EXAMPLES

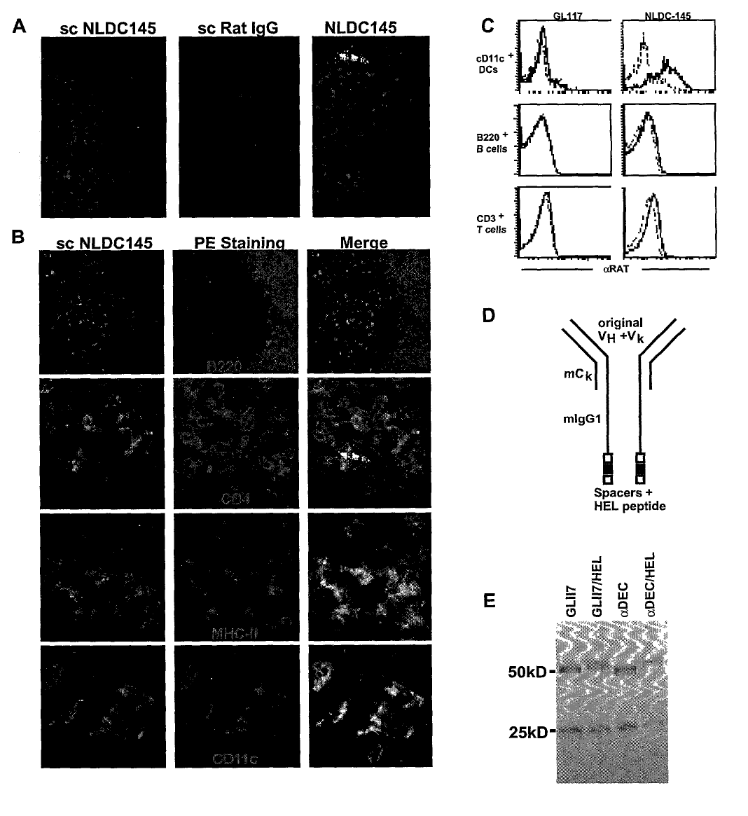

To determine whether the NLDC145 antibody targets DCs in vivo, mice were injected subcutaneously with purified NLDC145 or GL117, a non-specific isotype-matched rat monoclonal antibody control, and visualized the injected antibody in tissue sections. Popliteal lymph nodes (LNs) were removed from antibody-injected mice and 5 μm cryosections (Microm, Zeiss, Germany) were prepared. Tissue specimens were fixed in acetone (5 min, RT) air dried and stained in a moist chamber. The injected antibodies were detected by incubating the sections with streptavidin Cy3 or streptavidin-FITC (Jackson Immunotech). In double labeling experiments, the PE conjugated antibodies were added for additional 30 min. Specimens were examined using a fluorescence microscope and confocal optical sections of approx. 0.3 μm thickness were generated using deconvolution software (Metamorph). Twenty-four hours after injection, NLDC145 was found localized to scattered large dendritic profiles in the T cell areas of lymph nodes and spleen while uptake of control GL117 was undetectable (FIG. 1A left and middle). This pattern was similar to the pattern found when the antibody was applied to sections directly (FIG. 1A right). The NLDC145-targeted cells were negative for B220 and CD4, markers for B cells and T cells respectively, but positive for characteristic DC markers including MHC II and CD11c (FIG. 1B). Thus, subcutaneously injected NLDC145 targets specifically to CD11c⁺ MHC II⁺ DCs in lymphoid tissues in vivo. To further characterize the lymphoid cells that were targeted by NLDC145 in vivo, we stained lymphoid cell suspensions from antibody injected mice with anti-rat Ig and examined the cells by multiparameter flow cytometry (FIG. 1C). High levels of injected NLDC145 were found on the surface of most CD11c⁺ DCs but not on the surface of B220³⁰ B cells or CD3⁺ T cells (FIG. 1C). This shows that when NLDC145 is injected into mice it binds efficiently and directly to DCs but not to other lymphoid cells. To deliver antigens to DCs in vivo, fusion proteins were produced with amino acids 46-61 of hen egg lysozyme (HEL) added to the carboxyl terminus of cloned NLDC145 (αDEC/HEL) and GL117 (GL117/HEL) control antibody (FIG. 1D). Total RNA was prepared from NLDC-145 (C. Kurts, H. Kosaka, F. R. Carbone, J. F. Miller, W. R. Heath, J Exp Med 186, 239-45. (1997)) and GLII7 (gift of R. J. Hodes) hybridomas (both rat IgG2a) using Trizol (GibcoBRL). Full-length Ig cDNAs were produced with 5′-RACE PCR kit (GibcoBRL) using primers specific for 3′-ends of rat IgG2a and Ig kappa. The V regions were cloned in frame with mouse Ig kappa constant regions and IgG1 constant regions carrying mutations that interfere with FcR binding (K. Mahnke, et al., J Cell Biol 151, 673-84 (2000)). DNA coding for HEL peptide 46-61 with spacing residues on both sides was added to the C terminus of the heavy chain using synthetic oligonucleotides. Gene specific primers for cloning of rat IgG2a and Ig kappa:

| | |
|---|---|
| 5′ATAGTTTAGCGGCCCGCGATATCTCACTAACACTCATTCCTGTTGAAGCT | (SEQ ID NO:1); |
| 3′ATAGTTTAGCGGCCGCTCACTAGCTAGCTTTACCAGGAGAGTGGGAGAGAC | |
| TCTTCT | (SEQ ID NO:2). |

HEL peptide fragment construction:

| | |
|---|---|
| 5′CTAGCGACATGGCCAAGAAGGAGACAGTCTGGAGGCTCGAGGAGTTCGGT | (SEQ ID NO:3); |
| AGGTTCACAAACAGGAAC | |
| 5′ACAGACGGTAGCACAGACTATGGTATTCTCCAGATTAACAGCAGGTATTAT | (SEQ ID NO:4); |
| GACGGTAGGACATGATAGGC | |
| 3′GCTGTACCGGTTCTTCCTCTGTCAGACCTCCGAGCTCCTCAAGCCATCCAAG | (SEQ ID NO:5); |
| TGTTTGTCCTTGTGTCTG | |
| 3′CCATCGTGTCTGATACCATAAGAGGTCTAATTGTCGTCCATAATACTGCCAT | |
| CCTGTACTATCCGCCGG | (SEQ ID NO:6). |

To minimize antibody binding to Fc (FcR) receptors and further ensure the specificity of antigen targeting, the rat IgG2a constant regions of the original antibodies were replaced with mouse IgG1 constant regions that carry point mutations interfering with FcR binding (R. A. Clynes, T. L. Towers, L. G. Presta, J. V. Ravetch, Nat Med 6, 443-6 (2000)). The hybrid antibodies and control Igs without the terminal HEL (αDEC and GL117) were produced by transient transfection in 293 cells (FIG. 1E). Hybrid antibodies were transiently expressed in 293 cells after transfection using calcium phosphate. Cells were grown in serum free DMEM supplemented with Nutridoma SP (Boehringer). Antibodies were purified on Protein G columns (Pharmacia). The concentrations of purified antibodies were determined by ELISA using goat anti-mouse IgG1 (Jackson Immunotech).

Detailed description of FIG. 1: FIG. 1.NLDC-145 targets DCs in vivo. (A) Biotinylated NLDC-145 (scNLDC145 left) or rat IgG (scRatIgG middle) was injected into the hind footpads (50 μg/footpad) and inguinal lymph nodes harvested 24 hours later. Sections were stained with Streptavidin Cy3. Control sections from uninjected mice were stained using biotinylated NLDC145 and streptavidin Cy3 (NLDC145 right). (B) Two color immunofluorescence. Mice were injected with biotinylated NLDC145 as in (A) Sections were stained with streptavidin FITC (green) and PE-labeled antibodies (red) to B220 clone (RA3-6B2), CD4 (L3T4), MHC II (10-3.6), or CD11c clone (HL3) (all from PharMingen) as indicated. Specimens were analyzed by deconvolution microscopy. Double labeling is indicated by the yellow color. (C) FACS analysis of lymphoid cells after injection with NLDC145 and control GL117 antibody. B10.BR mice were injected subcutaneously in the footpads with 10 μg of NLDC145, or GL117 antibodies or PBS. Lymphoid cells were purified from peripheral lymph nodes 14 hours after antibody injection and stained with anti-rat IgG-RPE (Goat Anti-Rat IgG-RPE Serotec, UK) to visualize surface bound NLDC145 and GL117 antibodies. The cells were then incubated in mouse serum to block non-specific binding and stained with FITC anti-CD11c (HL3), or -B220 (RA3-6B2), or -CD3 (145-2C11); all antibodies were from PharMingen. Histograms show staining with anti-rat IgG on gated populations of CD 11c⁺ DCs, B220⁺ B cells and CD3⁺ T cells. (D) Diagrammatic representation of hybrid antibodies. Heavy and light chain constant regions of GL117 and NLDC145 monoclonal antibodies were replaced with mouse Ig kappa (mCk) and IgG1 constant (mIgG1) regions containing mutations that interfere with FcR binding. Sequences encoding the 46-61 HEL peptide with flanking spacer residues were added to the carboxyl ends of the heavy chains. (E) Hybrid antibodies. GL117, GL117/HEL, αDEC and αDEC/HEL antibodies analyzed by PAGE under reducing conditions, molecular weights in kD are indicated.

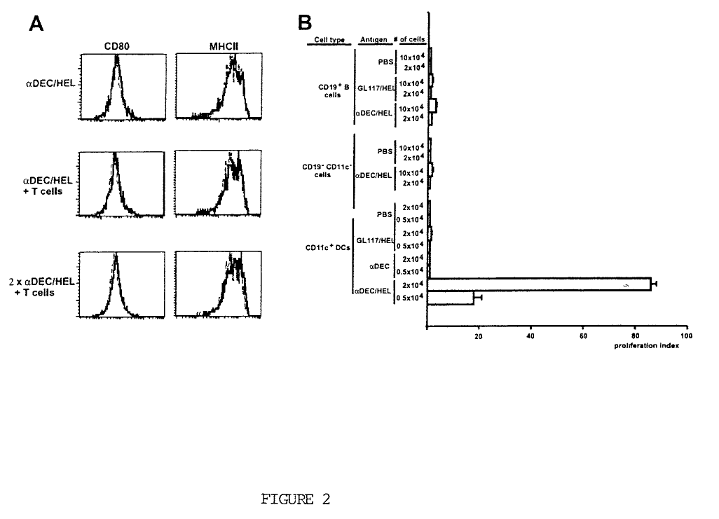

To determine whether antigens delivered by αDEC/HEL were processed by DCs in vivo, we injected mice with the hybrid antibodies and controls and tested CD11c⁺ DCs, CD19⁺ B cells and CD11c⁻ CD19⁻ mononuclear cells for their capacity to present HEL peptide to naïve HEL-specific T cells from 3A9 TCR transgenic mice (W. Y. Ho, M. P. Cooke, C. C. Goodnow, M. M. Davis, J Exp Med 179, 1539-49 (1994)). Six to 8 week old females were used in all experiments and were maintained under specific pathogen free conditions. B10.BR, B6.SJL (CD45.1) and B6/MRL (Fas lpr) mice were purchased from Jackson Laboratory. 3A9 transgenic mice were maintained by crossing with B10.BR mice. To obtain CD45.1 3A9 or 3A9/lpr T cells B6.SJL or B6/MRL mice were crossed extensively with 3A9 mice and tested for CD45.1 and I-Ak, by flow cytometry. Fas lpr mutation was tested by PCR. Mice were injected subcutaneously (s.c.) with peptide in CFA and s.c.or intravenously with chimeric antibodies. All experiments with mice were performed in accordance with NIH guidelines. DCs isolated from antibody-injected mice expressed levels of CD80 and MHC II similar to those found on PBS controls and thus showed no signs of increased maturation, in contrast to what occurs when DCs are stimulated with microbial products like bacterial lipopolysaccharide (LPS) and CpG deoxyoligonucleotides (T. De Smedt, et al., Journal of Experimental Medicine 184, 1413-24 (1996); T. Sparwasser, R. M. Vabulas, B. Villmow, G. B. Lipford, H. Wagner, European Journal of immunology 30, 3591-7 (2000)) (FIG. 2A). Nevertheless DCs from mice injected with αDEC/HEL induced strong T cell proliferative responses, whereas DCs isolated from PBS-injected mice or mice injected with the control antibodies had no effect (FIG. 2B). Pooled axillary, brachial, inguinal and popliteal lymph nodes were dissociated in 5% FCS RPMI and incubated in presence of collagenase (Boehringer) and EDTA as described (Hochrein et al., 2001, Differential production of IL-12, IFN-alpha, and IFN-gamma by mouse dendritic cell subsets. J Immunol 166:5448-55). For antigen presentation CD19+ and CD11c+ were purified using microbeads coupled to anti-mouse CD11c or CD19 IgG (Miltenyi) and irradiated with 1500 R. CD4 T cells were purified by depletion using rat antibodies supernatants specific for mouse: CD8 (TIB 211), B220 (RA3-6B2), MHC II (M5/114, TIB 120), F4/80 (F4/80,) and magnetic beads coupled to anti-rat IgG (Dynal). In antigen loading experiments the isolated presenting cells from each experimental group were cultured in 96-well plates with 2×10⁵ purified 3A9 CD4+ T cells. Cultures were maintained for 48 h with ³H-thymidine (1microCi) added for the last 6 h. The results were calculated as a ratio of proliferation in experimental groups to a PBS control group. The proliferation in PBS controls ranged from 500 to 2000 cpm.

For T cell proliferation assays in adoptive transfer recipients, 9×10⁴ of the same irradiated CD11c+ cells isolated from spleens of WT B10.BR mice were cultured in 96-well plates with 3×10⁵ T cells from each experimental group. Synthetic HEL peptide, at final concentration of 100 microgram/ml, was added to half of the cultures. Cultures were maintained for 24 h with ³H-thymidine (1microCi/ml) added for the last 6 h.

Response to HEL peptide was determined by subtracting background (no HEL peptide added) proliferation from proliferation in the presence of HEL peptide. Proliferation index was calculated as the ratio of the response to HEL peptide in a given experimental group to the response to HEL of T cells from a PBS injected control. Proliferation in PBS groups ranged from 4000-8000 cpm in the presence of peptide and the response to HEL peptide in these PBS controls was 1000-3000 counts above the background. Synthetic HEL 46-61 peptide was provided by the HHMI Keck Biotechnology Resource Center. DC isolated 3 days after αDEC/HEL injection showed reduced antigen-presenting activity (data not shown). In contrast to DCs, B cells and bulk CD11c⁻ CD19⁻ mononuclear cells purified from the same mice showed little antigen presenting activity (FIG. 2B). We conclude that antigens can be selectively and efficiently delivered to DC by αDEC/HEL in vivo, and the targeted DCs successfully process and load the peptides onto MHC II.

Detailed description of FIG. 2: DCs process and present antigen delivered by hybrid antibodies. (A) MHC II and CD80 expression on DCs is not altered by multiple injections of αDEC/HEL and 3A9 T cells. B10.BR mice transferred with 3A9 T cells and controls were injected subcutaneously in the footpads with 0.2 μg αDEC/HEL or PBS either at 8 days (αDEC/HEL) or at 1 and 8 days (αDEC/HELX2) after transfer (similar results were obtained by intravenous injection of chimeric antibodies—data not shown). Twenty-four hours after the last αDEC/HEL injection, DCs were purified from peripheral lymph nodes and analyzed by flow cytometry for expression of CD80 and MHC II (anti-CD80(B7-1)(16-10A1) ) and anti-I-A^{k} (10-3.6), respectively; PharMingen). Dotted lines in histograms indicate PBS control. (B) αDEC/HEL delivers HEL peptide to DCs in vivo. B10.BR mice were injected subcutaneously into footpads with 0.3 μg of αDEC/HEL or GL117/HEL or αDEC or PBS as indicated. CD11c⁺, CD19⁺ and CD11c⁻ CD19⁻ cells were isolated from draining lymph nodes 24 hours after antibody injection and assayed for antigen processing and presentation to purified 3A9 T cells in vitro. T cell proliferation was measured by ³H-thymidine incorporation and is expressed as a proliferation index relative to PBS controls. The results are means of triplicate cultures from one of four similar experiments.

Since DC isolation leads to activation, we performed adoptive transfer experiments with HEL-specific transgenic T cells to follow the response of these T cells to otherwise unmanipulated, antigen-targeted DC in vivo. CD4⁺ 3A9 T cells were transferred into B10.BR recipients. CD4 cells from 3A9 mice were enriched by depletion, washed 3× with PBS and 5×10⁶ cells injected intravenously per mouse. Alternatively, before depletion total cells were labeled with 2 μM CFSE in 5% FCS RPMI (Molecular Probes) at 37 C. for 20 min and washed twice and 24 h later hybrid antibodies were injected subcutaneously. To measure T cell responses, CD4⁺ cells were isolated from the draining lymph nodes of the injected mice and cultured in vitro in the presence or absence of added HEL peptide. Pooled axillary, brachial, inguinal and popliteal lymph nodes were dissociated in 5% FCS RPMI and incubated in presence of collagenase (Boehringer) and EDTA. For antigen presentation CD19+ and CD11c+ were purified using microbeads coupled to anti-mouse CD11c or CD19 IgG (Miltenyi) and irradiated with 1500 R. CD4 T cells were purified by depletion using rat antibodies supernatants specific for mouse: CD8 (TIB 211), B220 (RA3-6B2), MHC II (M5/114, TIB 120), F4/80 (F4/80,) and magnetic beads coupled to anti-rat IgG (Dynal). In antigen loading experiments the isolated presenting cells from each experimental group were cultured in 96-well plates with 2×10⁵ purified 3A9 CD4+ T cells. Cultures were maintained for 48 h with ³H-thymidine (1microCi) added for the last 6 h. The results were calculated as a ratio of proliferation in experimental groups to a PBS control group. The proliferation in PBS controls ranged from 500 to 2000 cpm.

For T cell proliferation assays in adoptive transfer recipients, 9×10⁴ of the same irradiated CD11c+ cells isolated from spleens of WT B10.BR mice were cultured in 96-well plates with 3×10⁵ T cells from each experimental group. Synthetic HEL peptide, at final concentration of 100 microg/ml, was added to half of the cultures. Cultures were maintained for 24 h with 3H-thymidine (1microCi/ml) added for the last 6 h. Response to HEL peptide was determined by subtracting background (no HEL peptide added) proliferation from proliferation in the presence of HEL peptide. Proliferation index was calculated as the ratio of the response to HEL peptide in a given experimental group to the response to HEL of T cells from a PBS injected control. Proliferation in PBS groups ranged from 4000-8000 cpm in the presence of peptide and the response to HEL peptide in these PBS controls was 1000-3000 counts above the background. Synthetic HEL 46-61 peptide was provided by the HHMI Keck Biotechnology Resource Center. T cell responses were measured by ³H-thymidine incorporation and are shown as proliferation indices normalized to the PBS control (this index facilitates comparison between experiments, see (31)). In addition to αDEC/HEL, GL117/HEL, αDEC and GL117 antibodies, we included 100 μg of HEL peptide in complete Freund's adjuvant (CFA) as a positive control.

As described in previous reports (E. R. Kearney, K. A. Pape, D. Y. Loh, M. K. Jenkins, Immunity 1, 327-39 (1994); L. Van Parijs, D. A. Peterson, A. K. Abbas, Immunity 8, 265-74 (1998)), CD4⁺ T cells isolated 2 days after challenge with 100 μg of HEL peptide in CFA showed strong proliferative responses to antigen when compared with PBS controls (FIG. 3A). Similar responses were obtained from mice injected with as little as 0.2 μg of αDEC/HEL (i.e., ˜4 ng peptide per mouse) but not from mice injected with up to 1 μg of αDEC, GL117 or GL117/HEL controls (FIG. 3A and not shown). We conclude that antigen delivered to DCs in vivo by αDEC/HEL efficiently induces activation of specific T cells.

To determine whether antigen delivered to DCs in vivo induces persistent T cell activation, we measured T cell responses to antigen 7 days after the administration of αDEC/HEL. CD4 T cells continued to show heightened responses to antigen when purified from LNs 7 days after injection with 100 μg of HEL peptide in CFA (E. R. Kearney, K. A. Pape, D. Y. Loh, M. K. Jenkins, Immunity 1, 327-39 (1994); L. Van Parijs, D. A. Peterson, A. K. Abbas, Immunity 8, 265-74 (1998)) (FIG. 3B). In contrast, T cells isolated from mice 7 days after injection with αDEC/HEL were no longer activated when compared to PBS controls (FIG. 3B). Thus, T cell activation by antigen delivered to DCs by αDEC/HEL in vivo is transient, readily detected at 2 but not 7 days. This transient activation resembles the CD4 T cell response to large doses of peptide in the absence of adjuvant, or the response to self antigens presented by bone marrow derived antigen presenting cells in the periphery (C. Kurts, H. Kosaka, F. R. Carbone, J. F. Miller, W. R. Heath, J Exp Med 186, 23945, (1997); D. J. Morgan, H. T. Kreuwel, L. A. Sherman, J Immunol 163, 723-7. (1999), E. R. Kearney, K. A. Pape, D. Y. Loh, M. K. Jenkins, Immunity 1, 327-39 (1994); L. Van Parijs, D. A. Peterson, A. K. Abbas, Immunity 8, 265-74 (1998); P. Aichele, K. Brduscha-Riem, R. M. Zinkernagel, H. Hengartner, H. Pircher, J Exp Med 182, 261-6 (1995)). To determine whether the absence of persistent T cell activation in mice injected with αDEC/HEL is due to clearance of the injected antigen, multiple doses of αDEC/HEL were administered. Repeated injection of αDEC/HEL at 3-day intervals failed to induce prolonged T cell activation (FIG. 3C). In addition, after 7 or 20 days, T cells initially activated by αDEC/HEL could not be re-activated when the mice were challenged with 100 μg of HEL peptide in CFA (FIG. 3D). Thus, the transient nature of the T cell response in mice injected with αDEC/HEL is not due to a lack of antigen, and T cells initially activated by DCs under physiologic conditions are unresponsive to subsequent challenge with antigen even in the presence of strong adjuvants.

Absence of persistent T cell responses could be due to DC deletion, T cell deletion, or induction of T cell anergy. To assess DC function in mice receiving multiple doses of αDEC/HEL, we isolated DCs from these mice and monitored presentation to 3A9 T cells in vitro (FIG. 3E) (see above methods). DCs from mice injected with two doses of antibody showed the same T cell stimulatory activity as DCs isolated from mice receiving a single injection of αDEC/HEL (FIG. 3E). In addition, the transfer of antigen specific T cells into αDEC/HEL recipients did not alter the ability of the isolated DCs to stimulate 3A9 T cells in vitro. Thus, the transient nature of the T cell response to DC-targeted-antigens in vivo is not the result of a lack of antigen-bearing DCs.

Detailed description of FIG. 3: In vivo activation of CD4⁺ T cells by αDEC/HEL. In all experiments, 3A9 T cells were transferred into B10.BR mice, and the recipients were injected subcutaneously in the footpads with antibodies in PBS or 100 μg of HEL peptide in CFA 24 hours after T cell transfer as indicated. T cell proliferation was measured by ³H-thymidine incorporation and is expressed as a proliferation index relative to PBS controls. (A) T cells are efficiently activated by antigen delivered by αDEC/HEL. 48 h after challenge with antigen in the indicated doses, CD4 T cells were isolated from peripheral lymph nodes and cultured in vitro with irradiated B10.BR CD11c⁺ cells in the presence or absence of HEL peptide. (B) CD4⁺ T cells are only transiently activated by antigen (αDEC/HEL 0.2 μg) delivered to DCs in vivo. CD4⁺ cells were purified from peripheral lymph nodes 2 or 7 days after challenge with antigen and cultured with irradiated CD11c⁺ cells in the presence or absence of HEL peptide. (C) Failure to induce persistent T cell activation with multiple injections of αDEC/HEL. 3A9 cells were transferred into B10.BR mice and recipients were injected with αDEC/HEL (0.2 μg/mouse) once (on day 9 or 2 before analysis) or multiple times (days 9, 6 and 2 before analysis). Assay for T cell activation was as above. (D) T cells initially activated by αDEC/HEL show diminished response to re-challenge with HEL peptide in CFA. Recipients were initially injected with either αDEC/HEL (0.2 μg), GL117/HEL(0.2 μg) or PBS and re-challenged 7 or 20 days later with 100 μg of HEL peptide in CFA or with PBS. CD4⁺ cells were purified from peripheral lymph nodes 2 days after the re-challenge and cultured with irradiated CD11c⁺ cells in the presence or absence of HEL peptide. Assay for T cell activation was as above. (E) Antigen loading of DCs with αDEC/HEL. B10.BR mice +/− transferred 3A9 T cells, were injected subcutaneously with 0.2 μg αDEC/HEL or PBS either at 8 days (αDEC/HEL) or at 1 and 8 days (αDEC/HELX2) after transfer. Antigen loading was measured 1 day after the last dose of αDEC/HEL by purifying CD11c⁺ DCs from peripheral lymph nodes and culturing with purified 3A9 T cells. The results are means of triplicate cultures from one of three similar experiments.

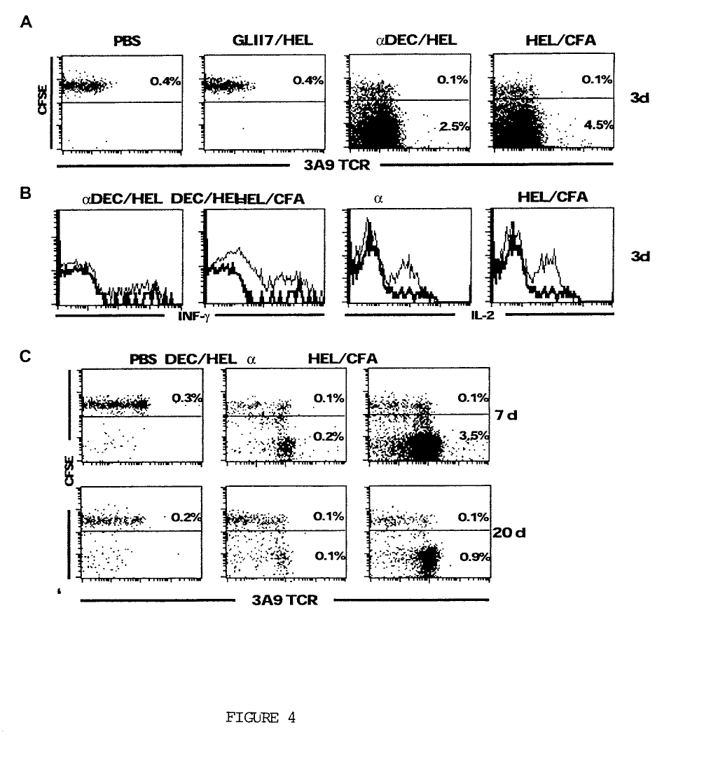

To examine the fate of 3A9 T cells after exposure to antigen presented by DCs in vivo, we performed adoptive transfer experiments with CD45.1⁺ 3A9 T cells labeled with 5-(6)-carboxyfluorescein diacetate succinimidyl diester (CFSE), a reporter dye for cell division. As previously described, T cells challenged with peptide in CFA divide, upregulate CD69 but not CD25 and produce IL-2 and IFNγ but not IL-4 or IL-10. These cells are therefore considered to be Th1 polarized (FIGS. 4A, B and not shown) (E. R. Kearney, K. A. Pape, D. Y. Loh, M. K. Jenkins, Immunity 1, 327-39 (1994); L. Van Parijs, D. A. Peterson, A. K. Abbas, Immunity 8, 265-74 (1998)). A burst of cell division and increase of CD69 but not CD25 expression was also seen after injection with 0.2 μg αDEC/HEL but not with GL117/HEL. Only clonotype positive CD4 cells showed these effects (FIGS. 4A, C and not shown). However, 3A9 cells activated by antigen presented on αDEC/HEL targeted DCs produced only IL-2 and no IFNγ, IL-4 or IL-10 and thus failed to polarize to Th1 or Th2 phenotype. (FIG. 4B and not shown). Therefore 3A9 cells proliferate in response to αDEC/HEL targeted DCs in vivo, but the T cells do not produce effector cytokines or polarize to Th1.

Although there was persistent expansion of 3A9 T cells in regional LNs and spleen 7 and 20 days after challenge with HEL peptide in CFA (FIG. 4C, spleen not shown), few 3A9 T cells survived in the LNs or spleen after exposure to antigen delivered by αDEC/HEL. The loss of 3A9 T cells was Fas independent as it also occurred with 3A9/lpr T cells (FIG. 4C). Thus, the initial expansion of T cells in response to antigen presented by DCs in vivo is not sustained, and most of the initial responding T cells disappear from lymphoid organs by day 7. These cells are either deleted or persist in extravascular sites (R. L. Reinhardt, A. Khoruts, R. Merica, T. Zell, M. K. Jenkins, Nature 410, 101-5 (2001). If they do persist outside lymphoid tissues they must be anergic, because they cannot be activated by further exposure to antigen, including peptide in CFA (FIG. 3D).

Detailed description of FIG. 4: CD4⁺ T cells divide in response to antigen presented by DCs in vivo, produce Il-2 but not IFN γ, and are then rapidly deleted. (A) CFSE labeled CD45.1⁺ 3A9 T cells were transferred into B10.BR and 24 hours later, the recipients were injected subcutaneously in the footpads with αDEC/HEL (0.2 μg), GL117/HEL (0.2 μg), HEL peptide in CFA or PBS. CD4⁺ T cells were purified by negative selection from regional lymph nodes. Three days after challenge with antigen they were analyzed by flow cytometry using antibodies specific for CD45.1 (A20), CD4 (L3T4) (both from PharMingen) and 3A9 T cell receptor (1G12). The plots show staining with 1G12 anti-3A9 and CFSE intensity on gated populations of CD4⁺CD45.1⁺ cells. The numbers indicate the percentage of CFSE high (undivided) and CFSE low (divided) CD4⁺ T cells. The results are from one of two similar experiments. (B) T cells produce Il-2 but not IFN-γ in response to antigens presented on DCs under physiological conditions. 3A9 cells were transferred into B10.BR mice and 24 hours later the recipients were injected subcutaneously in the footpads with αDEC/HEL (0.2 μg), GL117/HEL (0.2 μg), HEL peptide in CFA. CD4⁺. After 3 days T cells were purified by negative selection from regional lymph nodes as described in FIG. 3 and were stimulated for 4 hours with leukocyte activation cocktail (PharMingen). Cells were stained with antibodies specific for CD4 (L3T4) and 3A9 T cell receptor (1G12 ref). Fixed and permeabilized cells were then analyzed by flow cytometry using anti-IL-2-APC (JES6-5H4) and anti-IFN-γ-PE (XMG1.2) (PharMingen). Histograms show staining with anti-IL-2 and anti-IFN-γ on gated populations of 3A9⁺CD4⁺ cells. The thick lines indicate PBS control. (C) Same as in (A) but analysis performed 7 or 20 days after antigen administration.

DCs can be stimulated to increase their antigen presenting activity and their immunogenic potential by exposure to bacterial products or CD40L (C. Caux, et al., J Exp Med 180, 1263-72 (1994); K. Inaba, et al., J Exp Med 191, 927-36 (2000); F. Sallusto, A. Lanzavecchia, J Exp Med 1 19, 1109-18 (1994)), a TNF-family member expressed on activated CD4 T cells, platelets and mast cells (T. M. Foy, A. Aruffo, J. Bajorath, J. E. Buhlmann, R. J. Noelle, Annu Rev Immunol 14, 591-617 (1996)). To determine whether the combination of co-stimulators and antigen delivery to DCs produces persistent T cell activation, mice were injected with αDEC/HEL and the agonistic anti-CD40 antibody FGK 45 (A. Rolink, F. Melchers, J. Andersson, Immunity 5, 319-30 (1996)). In contrast to αDEC/HEL, the combination of αDEC/HEL and FGK 45 induced persistent T cell activation (FIG. 5B). The level of T cell activation seen with αDEC/HEL and FGK 45 at day 7 was comparable to αDEC/HEL at day 2 or HEL peptide in CFA at day 2 and 7 (compare FIGS. 3B and 5B). To determine whether anti-CD40 treatment altered 3A9 T cell numbers in αDEC/HEL treated mice, we performed adoptive transfer experiments with CD45.1 allotype-marked T cells and assayed by flow cytometry. Whereas FGK 45 alone showed no effect on the number of 3A9 T cells in LNs at day 7, the combination of FGK 45 and αDEC/HEL induced persistent ˜8-10 fold expansion of 3A9 T cells, an increase similar to that seen with HEL peptide in CFA at day 7 (FIG. 5A and FIG. 4). We conclude that persistent T cell responses can be induced by antigen delivered to DCs in vivo if an additional activation signal such as CD40 ligation is provided.

To determine if CD40 ligation induced detectable phenotypic changes on DCs in our system, we analyzed DCs from mice transferred with 3A9 cells and injected with FGK 45 and αDEC/HEL. Consistent with work by others we found that those DCs up-regulated their surface expression of CD40 and CD86 (FIG. 5C) (F. Koch, et al., Journal of Experimental Medicine 184, 741-6 (1996). This increase was more pronounced in the presence of antigen specific T cells suggesting a positive feedback mechanism between activated DCs and T cells (FIG. 5C).

Detailed description of FIG. 5: CD40 ligation prolongs T cell activation in response to antigens delivered to DCs and induces up-regulation of co-stimulatory molecules on DCs. (A) CD40 ligation induces persistent expansion of 3A9 cells in response to antigens delivered to DCs. CD45.1⁺3A9 T cells were transferred into B10.BR mice and 24 hours later the recipients were injected subcutaneously in the footpads with 0.2 μg of αDEC/HEL alone or 90 μg of FGK45 or both or PBS. CD4⁺ T cells were purified by negative selection from regional lymph nodes 7 days after challenge with antigen and analyzed by flow cytometry using antibodies specific for CD45.1 and CD4 as described in FIG. 4. The numbers indicate the percentages of CD4⁺ CD45.1⁺ cells in LNs. (B) CD40 ligation prolongs T cell activation. 3A9 T cells were transferred into B10.BR mice and 24 h later, recipients were injected subcutaneously in the footpads with 0.2 μg of αDEC/HEL alone or 90 μg of FGK45 or both or PBS. After 2 or 7 days, CD4 T cells were isolated from the draining lymph nodes and cultured in vitro with irradiated B10.BR CD11c⁺ cells in presence or absence of HEL peptide. T cell proliferation was measured by ³H-thymidine incorporation. The results represent triplicate cultures from two independent experiments. (C) CD40 ligation induces co-stimulatory molecules on DCs. B10.BR mice +/− 3A9 cell transfer were injected with 90 μg FGK45+0.2 μg αDEC/HEL or αDEC/HEL or PBS. 3 days later DCs were isolated as in FIG. 2 and analyzed by flow cytometry using antibodies specific for CD11c, B220, CD86 (GL1-biot) and CD40 (HM40-3-FITC) (all from PharMingen). Histograms show staining with anti-CD40 and anti-CD86 on gated populations of DCs. Thick lines indicate control with PBS, which was same as αDEC/HEL alone.

While the invention has been described and illustrated herein by references to the specific embodiments, various specific material, procedures and examples, it is understood that the invention is not restricted to the particular material combinations of material, and procedures selected for that purpose. Indeed, various modifications of the invention in addition to those described herein will become apparent to those skilled in the art from the foregoing description and the accompanying figures. Such modifications are intended to fall within the scope of the appended claims.

Various publications are cited herein, the disclosures of which are incorporated by reference in their entireties.

What is claimed is:

1. A method for enhancing the development of a cellular immune response to a preselected antigen in a mammal comprising exposing ex vivo or in vivo dendritic cells from said mammal to a conjugate comprising said preselected antigen covalently bound to an antibody to DEC-205, and promoting maturation of said dendritic cells ex vivo or in vivo by CD40 ligation.

2. The method of claim 1 wherein said preselected antigen is a peptide antigen or a protein antigen.

3. The method of claim 3 wherein said peptide antigen or protein antigen is conjugated to said antibody to DEC-205 by means of a cross-linking agent.

4. The method of claim 2 wherein a light chain or a heavy chain of said antibody to DEC-205, and said peptide antigen or protein antigen, are present on a single polypeptide chain.

5. The method of claim 1 wherein said CD40 ligation is achieved by exposing said dendritic cell to an agonistic anti-CD40 antibody.

6. A method for enhancing the development of tolerance to a preselected antigen in a mammal comprising exposing ex vivo or in vivo dendritic cells from said mammal to a conjugate comprising said preselected antigen covalently bound to an antibody to DEC-205, and preventing maturation of said dendritic cell ex vivo or in vivo.

7. The method of claim 6 wherein said preselected antigen is a peptide antigen or a protein antigen.

8. The method of claim 7 wherein said peptide or protein is conjugated to said antibody to DEC-205 by means of a cross-linking agent.

9. The method of claim 7 wherein a light chain or a heavy chain of said antibody to DEC-205, and said peptide antigen or protein antigen, are present on a single polypeptide chain.

10. A conjugate for enhanced delivery of a preselected protein or peptide antigen to a dendritic cell, said conjugate comprising said preselected protein or peptide antigen covalently bound to an antibody to DEC-205.

11. The conjugate of claim 10 wherein a light chain or a heavy chain of said antibody to DEC-205, and said peptide antigen or protein antigen, are present on a single polypeptide chain.

12. A method for enhancing the delivery of a preselected molecule into a dendritic cell comprising the steps of preparing a conjugate comprising said preselected molecule and an antibody to DEC-205, and exposing said conjugate to a dendritic cell, wherein said conjugate is delivered into said dendritic cell.
